(19) United States
(12) Patent Application Publication
(10) Pub. No.: US 2008/0306043 A1
(43) Pub. Date: Dec. 11, 2008

(54) Sabcomeline Alone or Combined with a Mood Stabilising or Antimanic Agent to Treat Bipolar Disorders

(76) Inventors: Carol Routledge, London (GB); James Joseph Hagan, Essex (GB); Stuart Paul Cuffe, Research Triangle Park, NC (US)

Correspondence Address: NIXON & VANDERHYE, PC, 901 NORTH GLEBE ROAD, 11TH FLOOR, ARLINGTON, VA, 22203, US

(21) Appl. No.: 11/792,850

(22) PCT Filed: Dec. 23, 2005
(86) PCT No.: PCT/GB05/05051

§ 371 Date: Aug. 22, 2008

(30) Foreign Application Priority Data
Dec. 23, 2004 (GB) 0428170.5

(51) Int. Cl. A61K 31/439; A61K 31/53; A61K 31/55; A61K 31/551; A61K 31/519; A61K 31/554; A61K 31/496; A61P 25/00

(52) U.S. Cl. 514/211.13; 514/305; 514/242; 514/217; 514/220; 514/259.41; 514/253.07; 514/254.04

(57) ABSTRACT

The invention relates to the use of sabcomeline or a pharmaceutically acceptable salt thereof in monotherapy for the treatment of bipolar disorder or mania or and to adjunctive and simultaneous combination therapies for the treatment of bipolar disorder or mania in which sabcomeline or a pharmaceutically acceptable salt thereof and at least one other mood stabilizing or antimanic agent are administered adjunctively or simultaneously. The invention provides methods of treatment of bipolar disorder or mania utilizing such monotherapy and such adjunctive or simultaneous therapeutic combination therapies, therapeutic combinations for use therein and pharmaceutical compositions comprising them.

This invention relates to monotherapy and combination therapy for treating bipolar disorders and mania, to therapeutic combinations and combinations comprising them for use in the treatment of bipolar disorders and mania, and to methods of treatment of bipolar disorders and mania.

U.S. Pat. No. 5,278,170 describes a class of compounds which enhance acetylcholine function via an action at muscarinic receptors within the central nervous system. A particularly preferred compound from within the scope of this disclosure has been given the common name sabcomeline, and has the following chemical structure (I)

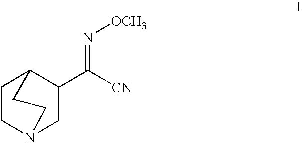

The chemical name for sabcomeline is R-(Z)-α-(methoxyimino)-α-(1-azabicyclo[2.2.2]oct-3-yl)acetonitrile. For therapeutic administration, it is preferably used in the form of a pharmaceutically acceptable salt, typically the hydrochloride salt, but alternative salts of sabcomeline with pharmaceutically acceptable acids may also be utilised in therapeutic administration, for example salts derived from sabcomeline free base and acids including, but not limited to, hydrobromic acid, phosphoric acid, acetic acid, furmaric acid, maleic acid, salicylic acid, citric acid, lactic acid, oxalic acid and p-toluene sulphonic acid.

Sabcomeline was initially evaluated for its use in the treatment of dementia. Subsequently, a number of disclosures have disclosed the use of sabcomeline for treating psychotic disorders, for example WO 98/46226; this disclosure does not, however disclose the use of sabcomeline for treating bipolar disorders, mania or manic depression. WO 02/03684 further discloses the treatment of psychotic disorders including bipolar disorders and mania by administration of a muscarinic agonist in combination with a typical or an atypical antipsychotic. Although sabcomeline is disclosed in WO 02/03684 as one of a number of muscarinic agonists suitable for combination with a large number of typical and atypical antipsychotics, exemplification is limited to just one muscarinic agonist (xanomeline) in combination with a small number of antipsychotics, and no specific information or data are recorded concerning combination therapy involving sabcomeline. Neither is any information provided which illustrates the activity of any muscarinic agonist combinations against bipolar disorders nor would enable the skilled reader to determine how to demonstrate such activity.

There remains a need to identify further and improved medicaments for use in the treatment of bipolar disorders and mania, and in particular compositions and methods of treatment which improve on the efficacy of existing therapies.

It has now been found that sabcomeline or a pharmaceutically acceptable salt thereof may advantageously be administered as monotherapy or in combination with at least one mood stabilising or antimanic agent to provide improved treatment of bipolar disorders and mania. Particular advantages associated with the combinations, uses and methods of treatment of the invention include equivalent or improved efficacy at doses of administration which are lower than those commonly used for the individual components where they are known for the treatment of bipolar disorders and mania. The combinations, uses and methods of treatment of the invention may also provide advantages in treatment of patients who fail to respond adequately or who are resistant to treatment with to certain mood stabilising or antimanic agents that are known for the treatment of bipolar disorders and mania.

For the avoidance of doubt, it is intended that the term bipolar disorder covers the full spectrum of bipolar disorders known to the skilled person.

For monotherapy, sabcomeline or a pharmaceutically acceptable salt thereof is administered independently of any other medication.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of sabcomeline or a pharmaceutically acceptable salt thereof and at least one mood stabilising or antimanic agent are within the scope of the current invention. In one embodiment of adjunctive therapeutic administration as described herein, a patient is typically stabilised on a therapeutic administration of one or more of the components for a period of time and then receives administration of another component. Within the scope of this invention, it is preferred that sabcomeline or a pharmaceutically acceptable salt thereof is administered as adjunctive therapeutic treatment to patients who are receiving administration of at least one mood stabilising or antimanic agent, but the scope of the invention also includes the adjunctive therapeutic administration of at least one mood stabilising or antimanic agent to patients who are receiving administration of sabcomeline or a pharmaceutically acceptable salt thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts. In a first aspect therefore, the invention provides a method of treatment of bipolar disorders or mania by administration of sabcomeline or a pharmaceutically acceptable salt thereof. In a further aspect, the invention provides the use of sabcomeline or a pharmaceutically acceptable salt thereof in the manufacture of a medicament for the treatment of bipolar disorders or mania. The invention also provides the use of sabcomeline or a pharmaceutically acceptable salt thereof for the treatment of bipolar disorders or mania. The invention further provides sabcomeline or a pharmaceutically acceptable salt thereof for use in the treatment of bipolar disorders or mania.

In a further aspect, the invention provides a method of treatment of bipolar disorders or mania by adjunctive therapeutic administration of sabcomeline or a pharmaceutically acceptable salt thereof to a patient receiving therapeutic administration of at least one mood stabilising or antimanic agent. In a further aspect, the invention provides the use of sabcomeline or a pharmaceutically acceptable salt thereof in the manufacture of a medicament for adjunctive therapeutic administration for the treatment of bipolar disorders or mania in a patient receiving therapeutic administration of at least one mood stabilising or antimanic agent. The invention also provides the use of sabcomeline or a pharmaceutically acceptable salt thereof in adjunctive therapeutic administration for the treatment of bipolar disorders or mania in a patient receiving therapeutic administration of at least one mood stabilising or antimanic agent. The invention also provides sabcomeline or a pharmaceutically acceptable salt thereof for use for adjunctive therapeutic administration for the treatment of bipolar disorders or mania in a patient receiving therapeutic administration of at least one mood stabilising or antimanic agent.

In a further aspect, the invention provides a method of treatment of bipolar disorders or mania by adjunctive therapeutic administration of at least one mood stabilising or antimanic agent to a patient receiving therapeutic administration of sabcomeline or a pharmaceutically acceptable salt thereof. In a further aspect, the invention provides the use of at least one mood stabilising or antimanic agent in the manufacture of a medicament for adjunctive therapeutic administration for the treatment of bipolar disorders or mania in a patient receiving therapeutic administration of sabcomeline or a pharmaceutically acceptable salt thereof. The invention also provides the use of at least one mood stabilising or antimanic agent for adjunctive therapeutic administration for the treatment of bipolar disorders or mania in a patient receiving therapeutic administration of sabcomeline or a pharmaceutically acceptable salt thereof.

In a further aspect, the invention provides a method of treatment of bipolar disorders or mania by simultaneous therapeutic administration of sabcomeline or a pharmaceutically acceptable salt thereof in combination with at least one mood stabilising or antimanic agent. The invention further provides the use of a combination of sabcomeline or a pharmaceutically acceptable salt thereof and at least one mood stabilising or antimanic agent in the manufacture of a medicament for simultaneous therapeutic administration in the treatment of bipolar disorders or mania. The invention further provides the use of a combination of sabcomeline or a pharmaceutically acceptable salt thereof and at least one mood stabilising or antimanic agent for simultaneous therapeutic administration in the treatment of bipolar disorders or mania. The invention further provides the use of sabcomeline or a pharmaceutically acceptable salt thereof in the manufacture of a medicament for simultaneous therapeutic administration with at least one mood stabilising or antimanic agent in the treatment of bipolar disorders or mania. The invention further provides the use of sabcomeline or a pharmaceutically acceptable salt thereof for simultaneous therapeutic administration with at least one mood stabilising or antimanic agent in the treatment of bipolar disorders or mania. The invention further provides sabcomeline or a pharmaceutically acceptable salt thereof for use for simultaneous therapeutic administration with at least one mood stabilising or antimanic agent in the treatment of bipolar disorders or mania. The invention further provides the use of at least one mood stabilising or antimanic agent in the manufacture of a medicament for simultaneous therapeutic administration with sabcomeline or a pharmaceutically acceptable salt thereof in the treatment of bipolar disorders or mania. The invention further provides the use of at least one mood stabilising or antimanic agent for simultaneous therapeutic administration with sabcomeline or a pharmaceutically acceptable salt thereof in the treatment of bipolar disorders or mania.

In further aspects, the invention provides a method of treatment of bipolar disorders or mania by simultaneous therapeutic administration of a pharmaceutical composition comprising sabcomeline or a pharmaceutically acceptable salt thereof and at least one mood stabilising or antimanic agent, a pharmaceutical composition comprising sabcomeline or a pharmaceutically acceptable salt thereof and at least one mood stabilising or antimanic agent, the use of a pharmaceutical composition comprising sabcomeline or a pharmaceutically acceptable salt thereof and at least one mood stabilising or antimanic agent for the treatment of bipolar disorders or mania, the use of a pharmaceutical composition comprising sabcomeline or a pharmaceutically acceptable salt thereof and at least one mood stabilising or antimanic agent in the manufacture of a medicament for the treatment of bipolar disorders or mania, and a pharmaceutical composition comprising sabcomeline or a pharmaceutically acceptable salt thereof and at least one mood stabilising or antimanic agent for use in the treatment of bipolar disorders or mania.

In a further aspect, the invention provides a kit-of-parts for use in the treatment of bipolar disorders or mania comprising a first dosage form comprising sabcomeline or a pharmaceutically acceptable salt thereof and one or more further dosage forms each comprising a mood stabilising or antimanic agent for simultaneous therapeutic administration.

Within the context of the present invention, the terms bipolar disorders and mania refer to all variations and sub-categories of bipolar disorder, mania, hypomania and manic depression including, without limitation, rapid cycling bipolar disorder and those categorised as shown below in “Diagnostic and Statistical Manual of Mental Disorders” (DSM-IV-TR), Fourth Edition, edited by American Psychiatric Association:

### 296.00 Bipolar I Disorder, Single Manic Episode, Unspecified

### 296.01 Bipolar I Disorder, Single Manic Episode, Mild

### 296.02 Bipolar I Disorder, Single Manic Episode, Moderate

296.03 Bipolar I Disorder, Single Manic Episode, Severe without Psychotic Features
296.04 Bipolar I Disorder, Single Manic Episode, Severe with Psychotic Features

### 296.05 Bipolar I Disorder, Single Manic Episode, In Partial Remission

### 296.06 Bipolar I Disorder, Single Manic Episode, In Full Remission

### 296.40 Bipolar I Disorder, Most Recent Episode Hypomanic

### 296.40 Bipolar I Disorder, Most Recent Episode Manic, Unspecified

### 296.41 Bipolar I Disorder, Most Recent Episode Manic, Mild

### 296.42 Bipolar I Disorder, Most Recent Episode Manic, Moderate

296.43 Bipolar I Disorder, Most Recent Episode Manic, Severe without Psychotic Features
296.44 Bipolar I Disorder, Most Recent Episode Manic, Severe with Psychotic Features

### 296.45 Bipolar I Disorder, Most Recent Episode Manic, In Partial Remission

### 296.46 Bipolar I Disorder, Most Recent Episode Manic, In Full Remission

### 296.50 Bipolar I Disorder, Most Recent Episode Depressed, Unspecified

### 296.51 Bipolar I Disorder, Most Recent Episode Depressed, Mild

### 296.52 Bipolar I Disorder, Most Recent Episode Depressed, Moderate

296.53 Bipolar I Disorder, Most Recent Episode Depressed, Severe without Psychotic Features
296.54 Bipolar I Disorder, Most Recent Episode Depressed, Severe with Psychotic Features

### 296.55 Bipolar I Disorder, Most Recent Episode Depressed, In Partial Remission

### 296.56 Bipolar I Disorder, Most Recent Episode Depressed, In Full Remission

### 296.60 Bipolar I Disorder, Most Recent Episode Mixed, Unspecified

### 296.61 Bipolar I Disorder, Most Recent Episode Mixed, Mild

### 296.62 Bipolar I Disorder, Most Recent Episode Mixed, Moderate

296.63 Bipolar I Disorder, Most Recent Episode Mixed, Severe without Psychotic Features
296.64 Bipolar I Disorder, Most Recent Episode Mixed, Severe with Psychotic Features

### 296.65 Bipolar I Disorder, Most Recent Episode Mixed, In Partial Remission

### 296.66 Bipolar I Disorder, Most Recent Episode Mixed, In Full Remission

### 296.80 Bipolar Disorder NOS

### 296.89 Bipolar II Disorder

All recognised forms and variations of the bipolar disorders mentioned herein are contemplated as within the scope of the present invention.

The treatment of bipolar disorder or mania with sabcomeline and a mood stabilising or antimanic agent as defined in the present invention may occur in addition to further drug therapies. In particular, tranquilizers may be used for the treatment of agitation, anxiety or sleep disturbances. Preferably lorazepam is used, which belongs to the class of benzodiazepines. Antidepressants and anxiolytics may also be used, for example SSRI antidepressants such as paroxetine or fluoxetine.

Examples of mood stabilising or antimanic agents that are useful in the present invention include those therapeutic agents known specifically for their mood stabilising or antimanic properties, such as lithium, but also include anti-convulsants having mood stabilising or antimanic plus antidepressant properties, such as lamotrigine, gabapentin, topirimate, valproate or carbamazepine, and certain neuroleptic (including typical antipsychotic and atypical antipsychotic) agents having mood stabilising or antimanic properties, including butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; dibenzoxazepines, such as loxapine; dihydroindolones, such as molindone; aripiprazole; and derivatives thereof that have antipsychotic activity.

It is believed that the clinical utility of the combination of sabcomeline and antipsychotic may vary between different members of the atypical antipsychotic drug class, depending on their different affinities for various sub-types of neurochemical receptors. For example, in addition to their affinities for dopamine and serotonin receptors, members of the atypical antipsychotic class may vary in their affinity for muscarinic and histamine receptor sub-types. The activity of atypical neuroleptics at muscarinic receptor subtypes are such that properties of negligible affinity, weak agonist activity and weak antagonist activity have been reported amongst the various members of the atypical antipsychotic drug class.

As an example, the M1/M4 receptor agonist properties of sabcomeline may enhance functional cholinergic activity and, when administered in combination, provide benefit by:

i) enhancing functional cholinergic activity in combination with an atypical antipsychotic that itself has little or no affinity for muscarinic receptors (e.g. risperidone)
ii) providing additive functional cholinergic activity in combination with an atypical antipsychotic drug that has weak muscarinic receptor agonist effects (e.g. clozapine or N-desmethylclozapine)
iii) competing for muscarinic receptors and thereby reducing the anticholinergic functional effects of an atypical antipsychotic drug that possesses muscarinic receptor antagonist properties (e.g. olanzepine).

As well as muscarinic and histaminergic receptors there are other receptors that may have benefit or adverse effects on cognition. For instance drugs with 5-HT6 receptor antagonist and adrenergic α2 receptor antagonist properties may also be of benefit. Some atypicals also have these benefits.

All references herein to mood stabilising or antimanic agents include references to other therapeutic agents having mood stabilising and antimanic properties, including anti-convulsant agents and neuroleptic (including typical and atypical antipsychotic) agents. Particular examples of mood stabilising and antimanic agents useful in the invention and their typical route of administration and dosage ranges that are preferred for use in the present invention are shown in Table 1.

TABLE 1

| Common | | Route of | | Dosage Range | |
|---|---|---|---|---|---|
| Name | Trade Name | Administration | Form | and (Median)^{a} | |
| Clozapine | CLOZARIL | oral | Tablets | 12.5-900 | mg/day |
| | | | | (300-900 | mg/day) |
| Olanzapine | ZYPREXA | oral | Tablets | 5-25 | mg/day |
| | | | | (10-25 | mg/day) |
| Ziprasidone | GEODON | oral | Capsules | 20-80 | mg/twice a day |
| | | | | (80-160 | mg/day) |
| Risperidone | RISPERDAL | oral | solution tablets | 2-16 | mg/day tablets |
| | | | | (4-12 | mg/day) |
| Risperidone | RISPERDAL | Intra-venous | Long-acting | | |
| | | | injectable form | | |
| Quetiapine | SEROQUEL | oral | Tablets | 50-900 | mg/day |
| fumarate | | | | (300-900 | mg/day) |
| Sertindole | SERDILECT | | | (4-24 | mg/day) |
| Amisulpride | | | | | |
| Haloperidol | HALDOL | oral | Tablets | 1-100 | mg/day |
| | | | | (1-15 | mg/day) |
| Haloperidol | HALDOL | parenteral | Injection | | |
| Decanoate | Decanoate | | | | |
| Haloperidol lactate | HALDOL | oral | Solution | | |
| | INTENSOL | parenteral | Injection | | |
| Chlorpromazine | THORAZINE | rectal | Suppositories | 30-800 | mg/day |
| | | oral | Capsules | (200-500 | mg/day) |
| | | | Solution | | |
| | | | Tablets | | |
| | | parenteral | Injection | | |
| Fluphenazine | PROLIXIN | | | 0.5-40 | mg/day |
| | | | | (1-5 | mg/day) |
| Fluphenazine | PROLIXIN | parenteral | Injection | (about one-half the | |
| Decanoate | Decanoate | | | dosage shown for oral) | |
| Fluphenazine | PROLIXIN | parenteral | Injection | (same as above | |
| Enanthate | | | | | |
| Fluphenazine | PROLIXIN | oral | Elixer | | |
| Hydrochloride | | | solution | | |
| | | parenteral | Injection | | |
| Thiothixene | NAVANE | oral | Capsules | 6-60 | mg/day |
| | | | | (8-30 | mg/day) |
| Thiothixene | NAVANE | oral | Solution | | |
| Hydrochloride | | parenteral | Injection | | |
| Trifluoperazine | STELAZINE | | | (2-40 | mg/day) |
| Perphenazine | TRILAFON | oral | Solution | 12-64 | mg/day |
| | | | Tablets | (16-64 | mg/day) |
| | | parenteral | Injection | | |
| Perphenazine and | ETRAFON | oral | Tablets | | |
| Amitriptyline | TRIAVIL | | | | |
| hydrochloride | | | | | |
| Thioridazine | MELLARIL | oral | Suspension | 150-800 | mg/day |
| | | | Solution | (100-300 | mg/day) |
| | | | tablets | | |
| Mesoridazine | | | | (30-400 | mg/day) |
| Molindone | MOBAN | | | 50-225 | mg/day |
| | | | | (15-150 | mg/day) |
| Molindone | MOBAN | oral | Solution | | |
| Hydrochloride | | | | | |
| Loxapine | LOXITANE | | | 20-250 | mg/day |
| | | | | (60-100 | mg/day) |
| Loxapine | LOXITANE | oral | solution | | |
| Hydrochloride | | parenteral | injection | | |
| Loxapine | LOXITANE | oral | capsules | | |
| Succinate | | | | | |
| Pimozide | | | | (1-10 | mg/day) |
| Flupenthixol | | | | | |
| Promazine | SPARINE | | | | |
| Triflupromazine | VESPRIN | | | | |
| Chlorprothixene | TARACTAN | | | | |
| Droperidol | INAPSINE | | | | |
| Acetophenazine | TINDAL | | | | |
| Prochlorperazine | COMPAZINE | | | | |
| Methotrimeprazine | NOZINAN | | | | |
| Pipotiazine | PIPOTRIL | | | | |
| Aripiprazole | | | | | |
| Hoperidone | | | | | |
| Lithium | | | | | |

Examples of tradenames and suppliers of selected mood stabilising, antimanic, anti-convulsant and neuroleptic agents are as follows: lamotrigine (available under the trade name LAMICTAL® from GlaxoSmithKline); valproate (DEPAKOTE®), carbamazepine (TEGRETOL®), gabapentin (NEURONTIN®), topirimate (TOPAMAX®) clozapine (available under the tradename CLOZARIL®, from Mylan, Zenith Goldline, UDL, Novartis); olanzapine (available under the tradename ZYPREXA®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from GlaxoSmithKline; fluphenazine (available under the tradename PROLIXIN®, from Apothecon, Copley, Schering, Teva, and American Pharmaceutical Partners, Pasadena); thiothixene (available under the tradename NAVANE®; from Pfizer); trifluoperazine (10-[3-(4-methyl-1-piperazinyl)propyl]-2-rifluoromethyl)phenothiazine dihydrochloride, available under the tradename STELAZINE®, from GlaxoSmithKline; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Particularly preferred mood stabilising or antimanic agents for use in the invention are lamotrigine, valproate, gabapentin, topiramate, oxcarbazepine, carbamazepine, olanzapine, risperidone, quetiapine, aripiprazole, haloperidol, clozapine, ziprasidone, lithium and osanetant.

A particularly preferred aspect of the invention provides a method of treatment of bipolar disorders or mania by adjunctive therapeutic administration of sabcomeline or a pharmaceutically acceptable salt thereof to a patient receiving administration of lamotrigine. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of lamotrigine. A further preferred aspect of the invention provides the use of sabcomeline or a pharmaceutically acceptable salt thereof for adjunctive therapeutic administration for the treatment of bipolar disorders or mania in a patient receiving administration of lamotrigine. A further preferred aspect of the invention provides sabcomeline or a pharmaceutically acceptable salt thereof for use for adjunctive therapeutic administration for the treatment of bipolar disorders or mania in a patient receiving administration of lamotrigine.

A particularly preferred aspect of the invention provides a method of treatment of bipolar disorders or mania by adjunctive therapeutic administration of sabcomeline or a pharmaceutically acceptable salt thereof to a patient receiving administration of lithium. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of lithium. A further preferred aspect of the invention provides the use of sabcomeline or a pharmaceutically acceptable salt thereof for adjunctive therapeutic administration for the treatment of bipolar disorders or mania in a patient receiving administration of lithium. A further preferred aspect of the invention provides sabcomeline or a pharmaceutically acceptable salt thereof for use for adjunctive therapeutic administration for the treatment of bipolar disorders or mania in a patient receiving administration of lithium.

A particularly preferred aspect of the invention provides a method of treatment of bipolar disorders or mania by adjunctive therapeutic administration of sabcomeline or a pharmaceutically acceptable salt thereof to a patient receiving administration of carbamazepine. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of carbamazepine. A further preferred aspect of the invention provides the use of sabcomeline or a pharmaceutically acceptable salt thereof for adjunctive therapeutic administration for the treatment of bipolar disorders or mania in a patient receiving administration of carbamazepine. A further preferred aspect of the invention provides sabcomeline or a pharmaceutically acceptable salt thereof for use for adjunctive therapeutic administration for the treatment of bipolar disorders or mania in a patient receiving administration of carbamazepine.

A particularly preferred aspect of the invention provides a method of treatment of bipolar disorders or mania by adjunctive therapeutic administration of sabcomeline or a pharmaceutically acceptable salt thereof to a patient receiving administration of valproate. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of valproate. A further preferred aspect of the invention provides the use of sabcomeline or a pharmaceutically acceptable salt thereof for adjunctive therapeutic administration for the treatment of bipolar disorders or mania in a patient receiving administration of valproate. A further preferred aspect of the invention provides sabcomeline or a pharmaceutically acceptable salt thereof for use for adjunctive therapeutic administration for the treatment of bipolar disorders or mania in a patient receiving administration of valproate.

A particularly preferred aspect of the invention provides a method of treatment of bipolar disorders or mania by adjunctive therapeutic administration of sabcomeline or a pharmaceutically acceptable salt thereof to a patient receiving administration of gabapentin. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of gabapentin. A further preferred aspect of the invention provides the use of sabcomeline or a pharmaceutically acceptable salt thereof for adjunctive therapeutic administration for the treatment of bipolar disorders or mania in a patient receiving administration of gabapentin. A further preferred aspect of the invention provides sabcomeline or a pharmaceutically acceptable salt thereof for use for adjunctive therapeutic administration for the treatment of bipolar disorders or mania in a patient receiving administration of gabapentin.

A particularly preferred aspect of the invention provides a method of treatment of bipolar disorders or mania by adjunctive therapeutic administration of sabcomeline or a pharmaceutically acceptable salt thereof to a patient receiving administration of topirimate. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of topirimate. A further preferred aspect of the invention provides the use of sabcomeline or a pharmaceutically acceptable salt thereof for adjunctive therapeutic administration for the treatment of bipolar disorders or mania in a patient receiving administration of topirimate. A further preferred aspect of the invention provides sabcomeline or a pharmaceutically acceptable salt thereof for use for adjunctive therapeutic administration for the treatment of bipolar disorders or mania in a patient receiving administration of topirimate.

A particularly preferred aspect of the invention provides a method of treatment of bipolar disorders or mania by adjunctive therapeutic administration of sabcomeline or a pharmaceutically acceptable salt thereof to a patient receiving administration of oxcarbazepine. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of oxcarbazepine. A further preferred aspect of the invention provides the use of sabcomeline or a pharmaceutically acceptable salt thereof for adjunctive therapeutic administration for the treatment of bipolar disorders or mania in a patient receiving administration of oxcarbazepine. A further preferred aspect of the invention provides sabcomeline or a pharmaceutically acceptable salt thereof for use for adjunctive therapeutic administration for the treatment of bipolar disorders or mania in a patient receiving administration of oxcarbazepine.

A particularly preferred aspect of the invention provides a method of treatment of bipolar disorders or mania by adjunctive therapeutic administration of sabcomeline or a pharmaceutically acceptable salt thereof to a patient receiving administration of olanzapine. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of olanzapine. A further preferred aspect of the invention provides the use of sabcomeline or a pharmaceutically acceptable salt thereof for adjunctive therapeutic administration for the treatment of bipolar disorders or mania in a patient receiving administration of olanzapine. A further preferred aspect of the invention provides sabcomeline or a pharmaceutically acceptable salt thereof for use for adjunctive therapeutic administration for the treatment of bipolar disorders or mania in a patient receiving administration of olanzapine.

A particularly preferred aspect of the invention provides a method of treatment of bipolar disorders or mania by adjunctive therapeutic administration of sabcomeline or a pharmaceutically acceptable salt thereof to a patient receiving administration of risperidone. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of risperidone. A further preferred aspect of the invention provides the use of sabcomeline or a pharmaceutically acceptable salt thereof for adjunctive therapeutic administration for the treatment of bipolar disorders or mania in a patient receiving administration of risperidone. A further preferred aspect of the invention provides sabcomeline or a pharmaceutically acceptable salt thereof for use for adjunctive therapeutic administration for the treatment of bipolar disorders or mania in a patient receiving administration of risperidone.

A particularly preferred aspect of the invention provides a method of treatment of bipolar disorders or mania by adjunctive therapeutic administration of sabcomeline or a pharmaceutically acceptable salt thereof to a patient receiving administration of quetiapine. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of quetiapine. A further preferred aspect of the invention provides the use of sabcomeline or a pharmaceutically acceptable salt thereof for adjunctive therapeutic administration for the treatment of bipolar disorders or mania in a patient receiving administration of quetiapine. A further preferred aspect of the invention provides sabcomeline or a pharmaceutically acceptable salt thereof for use for adjunctive therapeutic administration for the treatment of bipolar disorders or mania in a patient receiving administration of quetiapine.

A particularly preferred aspect of the invention provides a method of treatment of bipolar disorders or mania by adjunctive therapeutic administration of sabcomeline or a pharmaceutically acceptable salt thereof to a patient receiving administration of aripiprazole. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of aripiprazole. A further preferred aspect of the invention provides the use of sabcomeline or a pharmaceutically acceptable salt thereof for adjunctive therapeutic administration for the treatment of bipolar disorders or mania in a patient receiving administration of aripiprazole. A further preferred aspect of the invention provides sabcomeline or a pharmaceutically acceptable salt thereof for use for adjunctive therapeutic administration for the treatment of bipolar disorders or mania in a patient receiving administration of aripiprazole.

A particularly preferred aspect of the invention provides a method of treatment of bipolar disorders or mania by adjunctive therapeutic administration of sabcomeline or a pharmaceutically acceptable salt thereof to a patient receiving administration of haloperidol. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of haloperidol. A further preferred aspect of the invention provides the use of sabcomeline or a pharmaceutically acceptable salt thereof for adjunctive therapeutic administration for the treatment of bipolar disorders or mania in a patient receiving administration of haloperidol. A further preferred aspect of the invention provides sabcomeline or a pharmaceutically acceptable salt thereof for use for adjunctive therapeutic administration for the treatment of bipolar disorders or mania in a patient receiving administration of haloperidol.

A particularly preferred aspect of the invention provides a method of treatment of bipolar disorders or mania by adjunctive therapeutic administration of sabcomeline or a pharmaceutically acceptable salt thereof to a patient receiving administration of clozapine. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of clozapine. A further preferred aspect of the invention provides the use of sabcomeline or a pharmaceutically acceptable salt thereof for adjunctive therapeutic administration for the treatment of bipolar disorders or mania in a patient receiving administration of clozapine. A further preferred aspect of the invention provides sabcomeline or a pharmaceutically acceptable salt thereof for use for adjunctive therapeutic administration for the treatment of bipolar disorders or mania in a patient receiving administration of clozapine.

A particularly preferred aspect of the invention provides a method of treatment of bipolar disorders or mania by adjunctive therapeutic administration of sabcomeline or a pharmaceutically acceptable salt thereof to a patient receiving administration of ziprasidone. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of ziprasidone. A further preferred aspect of the invention provides the use of sabcomeline or a pharmaceutically acceptable salt thereof for adjunctive therapeutic administration for the treatment of bipolar disorders or mania in a patient receiving administration of ziprasidone. A further preferred aspect of the invention provides sabcomeline or a pharmaceutically acceptable salt thereof for use for adjunctive therapeutic administration for the treatment of bipolar disorders or mania in a patient receiving administration of ziprasidone.

A particularly preferred aspect of the invention provides a method of treatment of bipolar disorders or mania by adjunctive therapeutic administration of sabcomeline or a pharmaceutically acceptable salt thereof to a patient receiving administration of osanetant. A further preferred aspect of the invention provides the use of sabcomeline or a pharmaceutically acceptable salt thereof in the manufacture of a medicament for adjunctive therapeutic administration for the treatment of bipolar disorders or mania in a patient receiving administration of osanetant. A further preferred aspect of the invention provides the use of sabcomeline or a pharmaceutically acceptable salt thereof for adjunctive therapeutic administration for the treatment of bipolar disorders or mania in a patient receiving administration of osanetant. A further preferred aspect of the invention provides sabcomeline or a pharmaceutically acceptable salt thereof for use for adjunctive therapeutic administration for the treatment of bipolar disorders or mania in a patient receiving administration of osanetant.

For therapeutic administration according to the present invention, the sabcomeline monotherapy or the sabcomeline component of the combination therapy may be employed in the form of its free base, but is preferably used in the form of a pharmaceutically acceptable salt, typically the hydrochloride salt. Alternative salts of sabcomeline with pharmaceutically acceptable acids may also be utilised in therapeutic administration, for example salts derived from sabcomeline free base and acids including, but not limited to, hydrobromic acid, phosphoric acid, acetic acid, furmaric acid, maleic acid, salicylic acid, citric acid, oxalic acid, lactic acid, malic acid, methanesulphonic acid and p-toluene sulphonic acid. All solvates and all alternative physical forms of sabcomeline or its pharmaceutically acceptable derivatives as described herein, including but not limited to alternative crystalline forms, amorphous forms and polymorphs are also within the scope of this invention, and all references to sabcomeline herein include all pharmaceutically acceptable salts, and all solvates and alternative physical forms thereof.

The mood stabilising or antimanic agent component or components of the combination therapy may also be administered in their basic or acidic forms as appropriate or, where appropriate, in the form of a pharmaceutically acceptable salt or other derivative. All solvates and all alternative physical forms of the mood stabilising or antimanic agent or agents or their pharmaceutically acceptable salts or derivatives as described herein, including but not limited to alternative crystalline forms, amorphous forms and polymorphs, are also within the scope of this invention. In the case of the mood stabilising or antimanic agent or agents, the preferred forms and derivatives are those which are approved for therapeutic administration as monotherapies, including those mentioned in Table I, but all references to mood stabilising or antimanic agents herein include all pharmaceutically acceptable salts or other derivatives thereof, and all solvates and alternative physical forms thereof.

For therapeutic administration of the monotherapy component according to the invention, sabcomeline or its pharmaceutically acceptable salts or solvates may be administered in pure form, but will preferably be formulated into any suitable pharmaceutically acceptable and effective composition which provides effective levels of the active ingredient in the body. The choice of the most appropriate pharmaceutical compositions is within the skill of the art. Suitable formulations include, but are not limited to tablets, capsules, powders, granules, lozenges, suppositories, reconstitutable powders, or liquid preparations such as oral or sterile parenteral solutions or suspensions.

For adjunctive or simultaneous therapeutic administration according to the combination therapies of the invention, sabcomeline or its pharmaceutically acceptable salts or solvates and the mood stabilising or antimanic agent or agents or their pharmaceutically acceptable salts, derivatives or solvates may each be administered in pure form, but each of the components will preferably be formulated into any suitable pharmaceutically acceptable and effective composition which provides effective levels of the respective component in the body. The choice of the most appropriate pharmaceutical compositions for each component is within the skill of the art, and may be the same form or different forms for each of the components. Suitable formulations include, but are not limited to tablets, capsules, powders, granules, lozenges, suppositories, reconstitutable powders, or liquid preparations such as oral or sterile parenteral solutions or suspensions.

For simultaneous administration as a combined composition of sabcomeline or a pharmaceutically acceptable salt thereof and the mood stabilising or antimanic agent or agents according to the combination therapies of the invention, sabcomeline or its pharmaceutically acceptable salts or solvates and the mood stabilising or antimanic agent or agents and their pharmaceutically acceptable salts, derivatives or solvates may be administered together in pure form, but the combined components will preferably be formulated into any suitable pharmaceutically acceptable and effective composition which provides effective levels of each of the components in the body. The choice of the most appropriate pharmaceutical compositions for the combined components is within the skill of the art. Suitable formulations include, but are not limited to tablets, sub-lingual tablets, buccal compositions, capsules, powders, granules, lozenges, suppositories, reconstitutable powders, or liquid preparations such as oral or sterile parenteral solutions or suspensions.

In order to obtain consistency of administration of the monotherapy according to the invention and for consistency of adjunctive administration or simultaneous administration, it is preferred that the compositions used for the monotherapy component, the compositions of each of the components used for adjunctive therapy, and the compositions of the combination of the components used for simultaneous therapy are in the form of a unit dose.

Such unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling, tabletting or the like. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations for the monotherapy or for the components of the combination therapy, or for the combination of the components, may be in the form of, for example, emulsions, syrups, suspensions or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, or hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration (for example intravenous, intravascular or subcutaneous administration) of the monotherapy or of the components of the combination therapy, or of the combination of the components, fluid unit dosage forms are prepared utilizing the component or the combination of the components and a sterile vehicle, and, depending on the concentration used, can be either suspended or dissolved in the vehicle. In preparing solutions the monotherapy component, the components of the combination therapy or the combination of the components can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the component is suspended in the vehicle instead of being dissolved, and sterilization cannot be accomplished by filtration. The monotherapy component, the components of the combination therapy, or the combination of the components can be sterilized by exposure to ethylene oxide before suspending in the sterile vehicle. Advantageously, a surfactant or wetting agent is included in the composition to facilitate uniform distribution of the component or the combination of the components.

The monotherapy component, the components of the combination therapy or the combination of the components may also be formulated as depot preparations. Such long acting formulations may be administered by implantation (for example subcutaneously or intramuscularly) or by intramuscular injection. Thus, for example, the monotherapy component, the components of the combination therapy or the combination of the components of the invention may be formulated with suitable polymeric or hydrophobic materials (for example as an emulsion in an acceptable oil) or ion exchange resins, or as sparingly soluble derivatives, for example, as a sparingly soluble salt.

The compositions of the monotherapy component, or of each of the components or of the combination or of the components of the combination therapy may contain from 0.1% to 99% by weight, preferably from 10-60% by weight, of the active material, depending on the method of administration.

For monotherapy and for adjunctive or simultaneous administration, the unit dose of the sabcomeline component is in the range of 10-300 microgrammes, each unit dose being administered up to four times daily. Preferably the unit dose of the sabcomeline component is in the range 25-100 microgrammes, each unit dose being administered up to four times daily. For the combination therapies, the daily and unit doses of the mood stabilising or antimanic agent will depend upon which mood stabilising or antimanic agent is employed, but will typically be the recommended or approved dosage for the specific mood stabilising or antimanic agent when administered as monotherapy. In a preferred aspect of the invention, adjunctive administration of sabcomeline or a pharmaceutically acceptable salt thereof may permit lower doses of the mood stabilising or antimanic agent than those normally recommended when the mood stabilising or antimanic agent is prescribed as monotherapy. Typical daily doses of the mood stabilising or antimanic agents suitable for use in adjunctive or simultaneous administration according to the invention are shown in Table 1.

The monotherapy and the adjunctive or simultaneous administration of at least one mood stabilising or antimanic agent and sabcomeline or a pharmaceutically acceptable salt thereof as described herein may also be useful in the treatment or prevention of major depressive disorders including bipolar depression, unipolar depression, single or recurrent major depressive episodes with or without psychotic features, catatonic features, melancholic features, atypical features or postpartum onset, the treatment of anxiety and the treatment of panic disorders. Other mood disorders encompassed within the term major depressive disorders include dysthymic disorder with early or late onset and with or without atypical features, neurotic depression, post traumatic stress disorders, post operative stress and social phobia; dementia of the Alzheimer's type, with early or late onset, with depressed mood; vascular dementia with depressed mood; mood disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood. Major depressive disorders may also result from a general medical condition including, but not limited to, myocardial infarction, diabetes, miscarriage or abortion, etc.

The monotherapy or the adjunctive or simultaneous administration of at least one mood stabilising or antimanic agent and sabcomeline or a pharmaceutically acceptable salt thereof as described herein may also be useful in the treatment of sleep disorders including dysomnia, insomnia, sleep apnea, narcolepsy, and circadian rhythmic disorders.

The monotherapy and the adjunctive or simultaneous administration of at least one mood stabilising or antimanic agent and sabcomeline or a pharmaceutically acceptable salt thereof as described herein may also be useful in the treatment of tolerance to and dependence on a number of substances. For example, in the treatment of dependence on nicotine, alcohol, caffeine, phencyclidine (phencyclidine like compounds), or in the treatment of tolerance to and dependence on opiates (e.g. cannabis, heroin, morphine) or benzodiazepines; in the treatment of cocaine, sedative hypnotic, amphetamine or amphetamine—related drugs (e.g. dextroamphetamine, methylamphetamine) addiction or a combination thereof.

The invention may be illustrated by suitable patient studies. The following example of a suitable patient study is for illustrative purposes and is not intended to limit the scope of the invention in any way. The study is a multicentre, double-blind, randomized, parallel, placebo-controlled, 3-week inpatient comparison of 50 micrograms bid sabcomeline (as hydrochloride), 600 mg bid lithium, and placebo in subjects with Bipolar I Disorder (currently in a recurrent manic or mixed episode). To be eligible for enrollment, a subject must meet inclusion/exclusion requirements including: 1) having a diagnosis of Bipolar I Disorder and currently experiencing a Recurrent Manic or Mixed Episode (Appendices A and B, respectively) as defined in the Diagnostic and Statistical Manual of Mental Disorders, 4th edition, text revision (DSM-IV-TR) and based on the modified Structured Clinical Interview for Axis I DSM-IV Disorders (SCID) and 2) having a minimum of 20 on the YMRS. This study will last up to 42 days and will consist of 3 phases: a Screen Phase (2-7 days), a Treatment Phase (21 days), and a Follow-up Phase (14 days). After giving informed consent, completing the screening assessments, and meeting the inclusion/exclusion criteria, all subjects will enter a 2-7 day Screen Phase during hospitalization. This Phase will function: 1) as a washout period for other medications (if required) and 2) to discontinue subjects who do not continue to satisfy inclusion/exclusion criteria (e.g., based on clinical laboratory, physical examination, and/or ECG results). Following completion of the Screen Phase, subjects who continue to satisfy the inclusion/exclusion requirements will enter the 21-day Treatment Phase. Approximately 249 subjects will be randomized 1:1:1 to one of three treatment groups: 50 micrograms bid sabcomeline (as hydrochloride), 600 mg bid lithium, or placebo. The first dose of study medication will begin on the morning of Day 1 of the Treatment Phase. During the Treatment Phase, assessments will be conducted on Days 4, 7, 10, 14, 17, and 21. Subjects will remain in the hospital until the Day 7 assessments are completed. Subjects may leave the hospital anytime after completion of the Day 7 assessments if, in the Investigator's clinical judgement, they are ready for discharge and meet the community standards for level of functioning as an outpatient. Subjects who leave the hospital before Day 7 for any reason will be discontinued from the Treatment Phase, and study medication will be discontinued. The Follow-up Phase will permit safety to be assessed 14 days after the last dose of study medication. Efficacy will be assessed by using the YMRS, 21-item HAMD, CGI-S, CGI-I, and the GAF. Safety of the treatments will be evaluated by assessing vital signs, weights, clinical laboratory measures, ECGs, physical examinations, and adverse events.

1-40. (canceled)

41. A pharmaceutical composition comprising sabcomeline or a pharmaceutically acceptable salt thereof and at least one mood stabilising or antimanic agent.

42. A pharmaceutical composition according to claim 41 wherein the mood stabilising or antimanic agent is selected from the group consisting of lamotrigine, valproate, gabapentin, topiramate, oxcarbazepine, carbamazepine, olanzapine, risperidone, quetiapine, aripiprazole, haloperidol, clozapine, ziprasidone, lithium and osanetant.

43. A method of treatment of bipolar disorders by therapeutic administration of sabcomeline or a pharmaceutically acceptable salt thereof and at least one mood stabilising or antimanic agent to a patient.

44. A method of treatment according to claim 43 comprising adjunctive therapeutic administration of sabcomeline or a pharmaceutically acceptable salt thereof to a patient receiving therapeutic administration of at least one mood stabilising or antimanic agent.

45. A method of treatment according to claim 44 wherein the mood stabilising or antimanic agent is selected from the group consisting of lamotrigine, valproate, gabapentin, topiramate, oxcarbazepine, carbamazepine, olanzapine, risperidone, quetiapine, aripiprazole, haloperidol, clozapine, ziprasidone, lithium and osanetant.

46. A method of treatment according to claim 43 comprising adjunctive therapeutic administration of at least one mood stabilising or antimanic agent to a patient receiving therapeutic administration of sabcomeline or a pharmaceutically acceptable salt thereof.

47. A method of treatment according to claim 46 wherein the mood stabilising or antimanic agent is selected from the group consisting of lamotrigine, valproate, gabapentin, topiramate, oxcarbazepine, carbamazepine, olanzapine, risperidone, quetiapine, aripiprazole, haloperidol, clozapine, ziprasidone, lithium and osanetant.

48. A method of treatment according to claim 43 comprising simultaneous therapeutic administration of sabcomeline or a pharmaceutically acceptable salt thereof in combination with at least one mood stabilising or antimanic agent.

49. A method of treatment according to claim 48 wherein the mood stabilising or antimanic agent is selected from the group consisting of lamotrigine, valproate, gabapentin, topiramate, oxcarbazepine, carbamazepine, olanzapine, risperidone, quetiapine, aripiprazole, haloperidol, clozapine, ziprasidone, lithium and osanetant.
